(19) United States
(12) Patent Application Publication
(10) Pub. No.: US 2025/0232851 A1
(43) Pub. Date: Jul. 17, 2025

(54) SURVEILLANCE SYSTEMS, PLATFORMS, AND METHODS FOR AUTONOMOUS MONITORING OF CLINICAL STUDIES

(71) Applicant: KONINKLIJKE PHILIPS N.V., EINDHOVEN (NL)

(72) Inventors: Luoluo Liu, Cambridge, MA (US); Sara Mariani, Cambridge, MA (US); David Paul Noren, Cambridge, MA (US); David Craig McFarlane, Cambridge, MA (US)

(21) Appl. No.: 19/017,882

(22) Filed: Jan. 13, 2025

Related U.S. Application Data
(60) Provisional application No. 63/620,219, filed on Jan. 12, 2024.

(51) Int. Cl. G16H 10/20; G16H 50/70

(52) U.S. Cl. CPC G16H 10/20; G16H 50/70

(57) ABSTRACT

Provided herein are surveillance systems, platforms, and methods for autonomous monitoring of clinical studies that address these technical challenges and improve the credibility and validity of a clinical study by ensuring reliability of the data that is collected. In certain aspects, these systems, platforms, and methods assist in the communication between those responsible for analyzing the clinical study data (e.g., the data scientists, etc.) and those responsible for collecting the data (e.g., the physicians and clinical staff, etc.), allowing for proactive debugging of gaps in data coverage and data availability while there is still an opportunity to address the conditions of the clinical study leading to the missing data.

### STATEMENT AS TO FEDERALLY-SPONSORED RESEARCH

This invention was made with government support under a Notification of Toxic Exposure (NOTE) contract HQ0034209PT04, awarded by the Defense Threat Reduction Agency of the U.S. Department of Defense, and PREP contract HDTRA121C0006. The government has certain rights in the invention.

### FIELD OF THE DISCLOSURE

The present disclosure relates generally to surveillance systems and platforms, and more specifically to surveillance systems and platforms for autonomous monitoring of clinical studies.

### BACKGROUND

Clinical studies play an essential role in advancing our understanding of medical conditions and improving healthcare. These studies, often conducted with human or animal subjects, can provide important insights into the effects and/or progression of disease, can evaluate the effectiveness and safety of new treatments and/or medications, and can help develop new interventions and/or protocols. However, the reliability of data collected in clinical studies is critical to the credibility and validity of a clinical study. While rigorous methodologies, carefully designed protocols, and stringent quality control measures may be used, there are still challenges that impact the overall trustworthiness of the results. For example, challenges such as participant compliance, data interpretation, and unforeseen variables may arise. These and other challenges underscore the importance of robust review and statistical analysis of data collected in clinical studies.

### SUMMARY OF THE DISCLOSURE

It is appreciated by the present disclosure that the critical features of an observational clinical study can include data coverage and data availability. As described here, an observational clinical study includes observing a plurality of subjects using one or more different modalities or devices over one or more periods of time. In particular aspects, the observational clinical study may be linked to the occurrence of a specific event or type of event, such as the administration of a pharmaceutical compound prior to a medical procedure (e.g., administering an opioid prior to dental surgery, etc.).

In these and other clinical studies, the use of multiple devices applied simultaneously to the subjects creates unique technical challenges in reviewing and analyzing the data collected. For example, a variety of factors can contribute to gaps in time coverage and data availability, including, but not limited to, transmission errors from the clinical site, collection and/or quality errors due to device malfunction, as well as human errors (e.g., different practices to study protocols). Oftentimes, these gaps in a clinical study may not become evident until after all of the data is collected. Further, it should be appreciated that the individuals (e.g., data scientists, researchers, etc.) reviewing and analyzing the data collected in a clinical study may not be the same individuals responsible for collecting the data (e.g., physicians and clinical staff, etc.). Thus, there is limited ability to correct data coverage and data availability gaps ex post facto.

Accordingly, provided herein are surveillance systems, platforms, and methods for autonomous monitoring of clinical studies that address these technical challenges and improve the credibility and validity of a clinical study by ensuring reliability of the data that is collected. In further aspects, the systems, platforms, and methods described herein assist communication between those responsible for analyzing the clinical study data (e.g., the data scientists, etc.) with those responsible for collecting the data (e.g., the physicians and clinical staff, etc.), allowing for proactive debugging of gaps in data coverage and data availability while there is still an opportunity to address the conditions of the clinical study leading to the missing data.

According to an embodiment of the present disclosure, a method of dynamically monitoring incoming clinical study data is provided. The method can include: obtaining a clinical study dataset comprising physiological information for a plurality of subjects over a period of time, wherein the physiological information includes two or more measurements collected from the plurality of subjects during the period of time using two or more different devices; analyzing the clinical study dataset to synchronize the two or more measurements collected for each subject according to a common clock; determining two or more data coverage metrics for the clinical study dataset based on the synchronized measurements, wherein each data coverage metric corresponds to one of the two or more different devices; determining two or more collection duration metrics for the clinical study dataset based on the synchronized measurements, wherein each collection duration metric corresponds to either (i) one of the two or more different devices, or (ii) one of the two or more measurements; generating a dynamic graphical user interface including the two or more data coverage metrics and the two or more collection duration metrics determined for the clinical study dataset; displaying, on a display device, the dynamic graphical user interface.

In an aspect, determining two or more data coverage metrics can include: determining a first data coverage metric for a first device of the two or more different devices, wherein the first data coverage metric corresponds to a percentage of the plurality of subjects for whom the physiological information contains measurements collected using the first device; and determining a second data coverage metric for a second device of the two or more different devices, wherein the second data coverage metric corresponds to a percentage of the plurality of subjects for whom the physiological information contains measurements collected using the second device.

In an aspect, determining two or more duration coverage metrics can include, for each subject of the plurality of subjects: determining a first collection duration metric for a first device of the two or more different devices, wherein the first collection duration includes a first start time that corresponds to when the first device began collecting measurements from the subject, and a first end time that corresponds to when the first device stopped collecting measurements from the subject; and determining a second collection duration metric for a second device of the two or more different devices, wherein the second collection duration includes a second start time that corresponds to when the second device began collecting measurements from the subject, and a second end time that corresponds to when the second device stopped collecting measurements from the subject.

In an aspect, determining two or more duration coverage metrics can include, for each subject of the plurality of subjects: determining a first collection duration metric for a first measurement of the two or more measurements, wherein the first collection duration includes a first start time that corresponds to when one of the two or more different devices began collecting the first measurement from the subject, and a first end time that corresponds to when the device stopped collecting the first measurement; and determining a second collection value for a second measurement of the two or more measurements, wherein the second collection duration includes a second start time that corresponds to when one of the two or more different devices began collecting the second measurement from the subject, and a second end time that corresponds to when the device

In an aspect, the same device may be used to collect the first measurement and the second measurement.

In an aspect, the two or more data coverage metrics and the two or more collection duration metrics may be determined relative to the occurrence of at least one event of interest.

In an aspect, the method can further include: receiving, via an input device, user input identifying the at least one event of interest.

According to another embodiment of the present disclosure, a system for dynamically monitoring incoming clinical study data is provided. The system can include: a display device configured to display a dynamic graphical user interface; an input device configured to receive user input; a computer-readable storage medium having stored thereon computer-readable instructions to be executed by one or more processors; and one or more processors configured by the computer-readable instructions stored on the computer-readable storage medium to perform the following operations: (i) obtain a clinical study dataset comprising physiological information for a plurality of subjects over a period of time, wherein the physiological information includes two or more measurements collected from the plurality of subjects during the period of time using two or more different devices; (ii) analyze the clinical study dataset to synchronize the two or more measurements collected for each subject according to a common clock; (iii) determine two or more data coverage metrics for the clinical study dataset based on the synchronized measurements, wherein each data coverage metric corresponds to one of the two or more different devices; (iv) determine two or more collection duration metrics for the clinical study dataset based on the synchronized measurements, wherein each collection duration metric corresponds to either (a) one of the two or more different devices, or (b) one of the two or more measurements; (v) generate a dynamic graphical user interface including the two or more data coverage metrics and the two or more collection duration metrics determined for the clinical study dataset; and (vi) display, on a display device, the dynamic graphical user interface.

In an aspect, the one or more processors can be further configured by the computer-readable instructions stored on the computer-readable storage medium to perform the following operations: receive, via the input device, user input identifying at least one event of interest; and determine the two or more data coverage metrics and the two or more collection duration metrics relative to the occurrence of the at least one event of interest.

In an aspect, the dynamic graphical user interface can include: a first graphical module including a graphical representation of the two or more data coverage metrics for the clinical study dataset; and a second graphical module including a graphical representation of the two or more collection duration metrics for the clinical study dataset.

In an aspect, the dynamic graphical user interface can include: a third graphical module including a graphical representation of the physiological information available for the plurality of subjects stratified along a first axis corresponding to a start timestamp of the physiological information offset relative to an event of interest, and along a second axis corresponding to an end timestamp of the physiological information offset relative to the event of interest, wherein each subject is represented by a selectable indicator.

In an aspect, the one or more processors can be further configured by the computer-readable instructions stored on the computer-readable storage medium to perform the following operations: receive, via the input device, a user selection of a selectable indicator corresponding to a selected subject; and updating the third graphical module to present detailed information for the selected subject, wherein the detailed information includes physiological information for the selected subject not represented in the third graphical module.

According to another embodiment of the present disclosure, a computer program product can include a computer-readable storage medium having stored thereon computer-readable instructions that, when executed by one or more processors, cause the one or more processors to perform the following operations: (i) obtain a clinical study dataset comprising physiological information for a plurality of subjects over a period of time, wherein the physiological information includes two or more measurements collected from the plurality of subjects during the period of time using two or more different devices; (ii) analyze the clinical study dataset to synchronize the two or more measurements collected for each subject according to a common clock; (iii) determine two or more data coverage metrics for the clinical study dataset based on the synchronized measurements, wherein each data coverage metric corresponds to one of the two or more different devices; (iv) determine two or more collection duration metrics for the clinical study dataset based on the synchronized measurements, wherein each collection duration metric corresponds to either (a) one of the two or more different devices, or (b) one of the two or more measurements; (v) generate a dynamic graphical user interface including the two or more data coverage metrics and the two or more collection duration metrics determined for the clinical study dataset; and (vi) display, on a display device, the dynamic graphical user interface.

In an aspect, the computer-readable storage medium may include computer-readable instructions to determine two or more data coverage metrics by: determine a first data coverage metric for a first device of the two or more different devices, wherein the first data coverage metric corresponds to a percentage of the plurality of subjects for whom the physiological information contains measurements collected using the first device; and determine a second data coverage metric for a second device of the two or more different devices, wherein the second data coverage metric corresponds to a percentage of the plurality of subjects for whom the physiological information contains measurements collected using the second device.

In an aspect, the computer-readable storage medium may include computer-readable instructions to determine two or more duration coverage metrics by, for each subject of the plurality of subjects: determine a first collection duration metric for a first device of the two or more different devices, wherein the first collection duration includes a first start time that corresponds to when the first device began collecting measurements from the subject, and a first end time that corresponds to when the first device stopped collecting measurements from the subject; and determine a second collection duration metric for a second device of the two or more different devices, wherein the second collection duration includes a second start time that corresponds to when the second device began collecting measurements from the subject, and a second end time that corresponds to when the second device stopped collecting measurements from the subject.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart illustrating a method of dynamically monitoring incoming clinical study data in accordance with aspects of the present disclosure.

FIG. 2 is a diagram illustrating the collection of data in a clinical study in accordance with aspects of the present disclosure.

FIG. 3 is a diagram illustrating the collection of data in a clinical study in accordance with further aspects of the present disclosure.

FIG. 4 is a block diagram illustrating a time-series synchronization framework in accordance with aspects of the present disclosure.

FIG. 5 is a graph illustrating the abnormality location detection between two time-series in accordance with aspects of the present disclosure.

FIG. 6 is a block diagram illustrating a system for dynamically monitoring incoming clinical study data in accordance with aspects of the present disclosure.

FIG. 7A is an illustration of a dynamic graphical user interface having a first type of graphical module in accordance with aspects of the present disclosure.

FIG. 7B is an illustration of a dynamic graphical user interface having a second type of graphical module in accordance with aspects of the present disclosure.

FIG. 7C is an illustration of a dynamic graphical user interface having a third type of graphical module in accordance with aspects of the present disclosure.

FIG. 7D is an illustration of a dynamic graphical user interface having a fourth type of graphical module in accordance with aspects of the present disclosure.

FIG. 7E is an illustration of a dynamic graphical user interface having a fifth type of graphical module in accordance with aspects of the present disclosure.

FIG. 7F is an illustration of a dynamic graphical user interface having a sixth type of graphical module in accordance with aspects of the present disclosure.

### DETAILED DESCRIPTION OF EMBODIMENTS

Provided herein are surveillance systems, platforms, and methods for autonomous monitoring of clinical studies that address a variety of technical challenges and improve the credibility and validity of a clinical study by ensuring reliability of the data that is collected. In particular aspects, the embodiments described herein assist in the communication between those responsible for analyzing the clinical study data (e.g., the data scientists, etc.) and those responsible for collecting the data (e.g., the physicians and clinical staff, etc.), thereby allowing for proactive debugging of gaps in data coverage and data availability while there is still an opportunity to address the conditions of the clinical study leading to the missing data. In specific aspects, the embodiments described herein enable the dynamic tracking of clinical study performance through a number of different user interface modules.

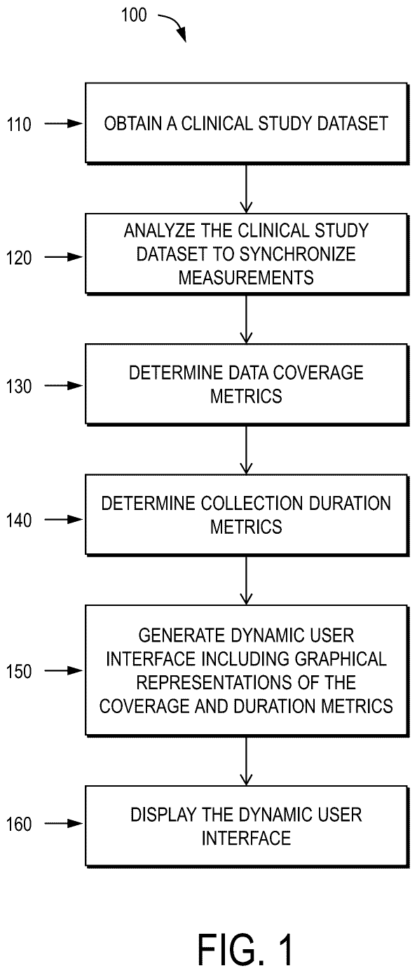

For example, with reference to FIG. 1, a method 100 of dynamically monitoring incoming clinical study data is illustrated in accordance with various aspects of the present disclosure. As shown, the method 100 can include: in a step 110, obtaining a clinical study dataset; in a step 120, analyzing the clinical study dataset to synchronize different measurements; in a step 130, determining data coverage metrics for the clinical study dataset; in a step 140, determining collection duration metrics for the clinical study dataset; in a step 150, generating a dynamic graphical user interface including graphical representations of the data coverage and collection duration metrics; and in a step 160, displaying the dynamic user interface.

More specifically, in the step 110, the method 100 can include obtaining a clinical study dataset comprising physiological information and other data for a plurality of subjects over a period of time. As described herein, the physiological information can include, but is not limited to, physiological measurements (heart rate, respiratory rate, blood oxygen saturation, etc.), as well as test results related to cells, tissues, body fluids, or imaging of the subjects 100A-D. In embodiments, the physiological information includes multiple measurements collected from the plurality of subjects during the period of time using multiple devices. In particular embodiments, the physiological information may include two or more measurements collected from the plurality of subjects during the period of time using two or more different devices, depending on the setup of the clinical study.

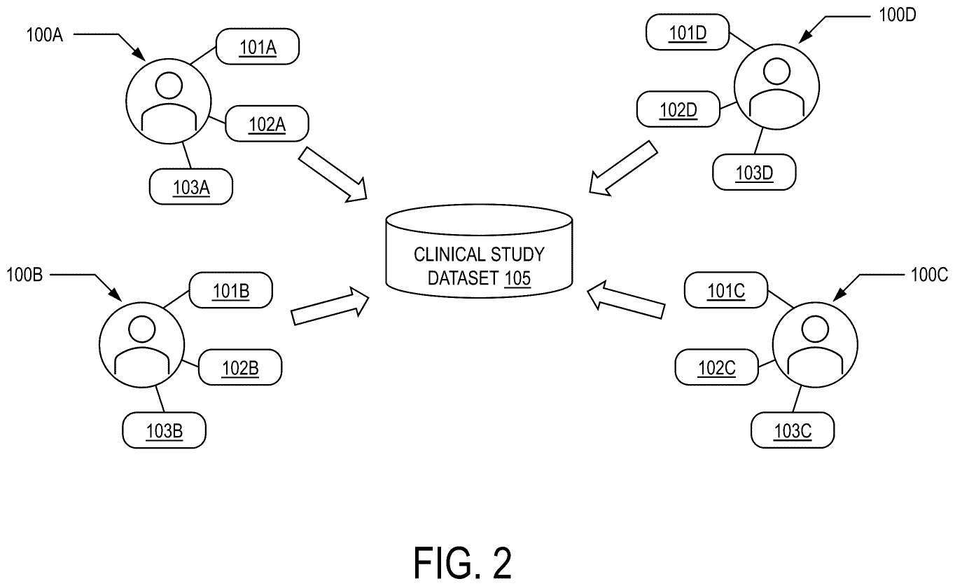

As shown in the example of FIG. 2, an exemplary setup of an observational clinical study of a plurality of subjects 100A-D is illustrated in accordance with certain aspects of the present disclosure. In particular, each of the plurality of subjects 100A-D may be associated with one or more care-related devices 101A-D, 102A-D, 103A-D. As shown, each subject 100A-D is monitored using three different devices 101A-D, 102A-D, 103A-D, such as a first device 101A-D, a second device 102A-D, and a third device 103A-D. In some embodiments, the devices 101A-D, 102A-D, 103A-D can include, for example, a multi-function patient monitor, a wearable device having one or more physiological sensors, and/or the like. In specifical embodiments, the devices 101A-D, 102A-D, 103A-D can include, but is not limited to, a Philips patient monitor, an Empatica wristband, Oura ring, Bio-tel ePatch, Dexcom glucose and lactate sensors, FinaPres Nova monitor, and/or the like.

Although three different devices 101A-D, 102A-D, 103A-D are illustrated in FIG. 2, it should be appreciated that fewer devices (i.e., one or two devices) may be used, as well as more devices (i.e., more than 3 devices). Furthermore, it should be appreciated that the number and type of devices 101A-D, 102A-D, 103A-D used to monitor a subject 100A-D may be different for each subject 100A-D. For example, as discussed in more detail below, the clinical study dataset 105 may include physiological information collected by one or two devices 101A-D, 102A-D, 103A-D for a first subject, whereas the clinical study dataset 105 includes physiological information collected by three or more devices 101A-D, 102A-D, 103A-D for a second subject.

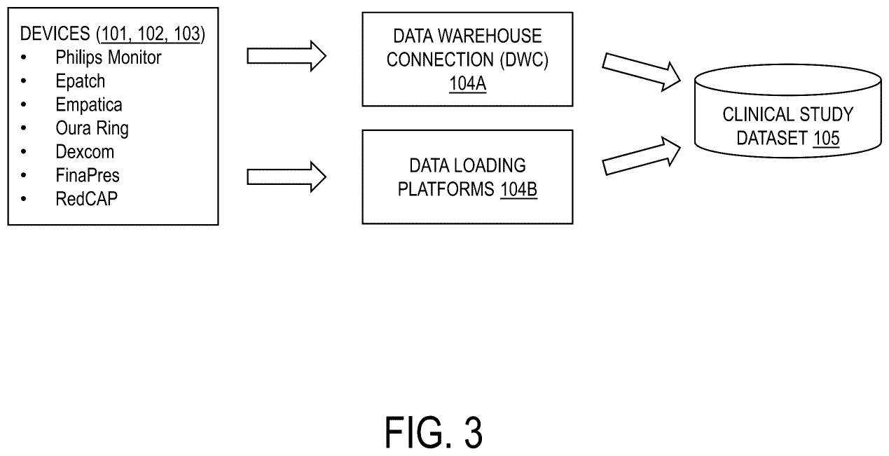

With reference to FIG. 3, the relevant physiological information may be collected from the devices 101A-D, 102A-D, 103A-D through an intermediary platform or system, such as a data warehouse connection (DWC) 104A and/or a data loading platform 104B. Thus, in particular embodiments, the clinical study dataset 105 may include information collected directly and/or indirectly from the devices 101A-D, 102A-D, 103A-D.

According to the present disclosure, the clinical study dataset may include physiological information collected for each of a plurality of subjects at different points in time, thereby encompassing a larger period of time. For example, a clinical study may observe certain aspects of a dental surgery practice over a one-year period, during which the practice operates on five new patients every month. Under this particular clinical study, each patient may be observed 24-48 hours prior to receiving dental surgery and 24-48 hours after receiving dental surgery. Thus, a total of 60 patients are observed over the one-year period of time, and the physiological information collected for the patients 100A-D spans the one-year period. However, the data coverage and collection duration metrics may refer to a clinically-relevant period of time (e.g., 24-48 hours before and/or after the event of interest), rather than this one-year period of time.

Next, in the step 120, the method 100 can include analyzing the clinical study dataset 105 to synchronize the measurements collected for each subject according to a common clock. In other words, the clinical study dataset 105 may be pre-processed to at least calculate the start and end times of outputs from the devices 101A-D, 102A-D, 103A-D according to a common timescale/clock. It should be appreciated that each device 101A-D, 102A-D, 103A-D may operate according to its own internal timing mechanism (i.e., clock), and may or may not include references to a standard or common clock. Some devices (such as an Oura ring) may output physiological information output for a subject (e.g., beat annotations in the sleep and rest periods temperature measurements during wear time) containing multiple timestamps based on different timing mechanisms.

In further examples, the devices 101A-D, 102A-D, 103A-D may provide outputs such that timestamps are not directly findable from meta files. For example, monitoring timestamps from a device 101A-D, 102A-D, 103A-D with different channels (such as heart rate and respiratory rate) may not be directly findable from meta files, and therefore pre-processing steps are required to determine the start and end times for the outputs of each channel.

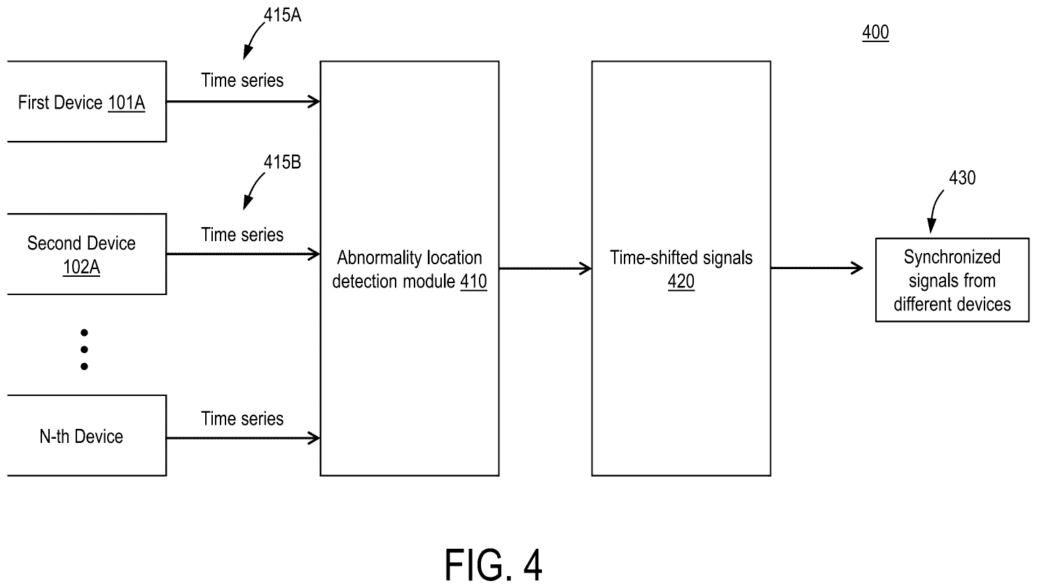

Accordingly, in embodiments, the systems and methods described herein may include a framework 400 for synchronizing the measurements collected for each subject according to a common clock. For example, as shown in FIG. 4, the framework 400 can include an abnormality location detection module 410 that receives at least a first physiological measurement time series from two or more devices (e.g., devices 101A, 102A, etc.). In embodiments, each time series 415A, 415B may correspond to the same physiological parameter and be input to the module 410 from different devices 101A, 102A. In further embodiments, the time-series 415A, 415B can be raw (for example, inter-beat intervals series, temperature, etc.), or it can be derived features (for example, derived averaged heart rate from ECG or PPG signals).

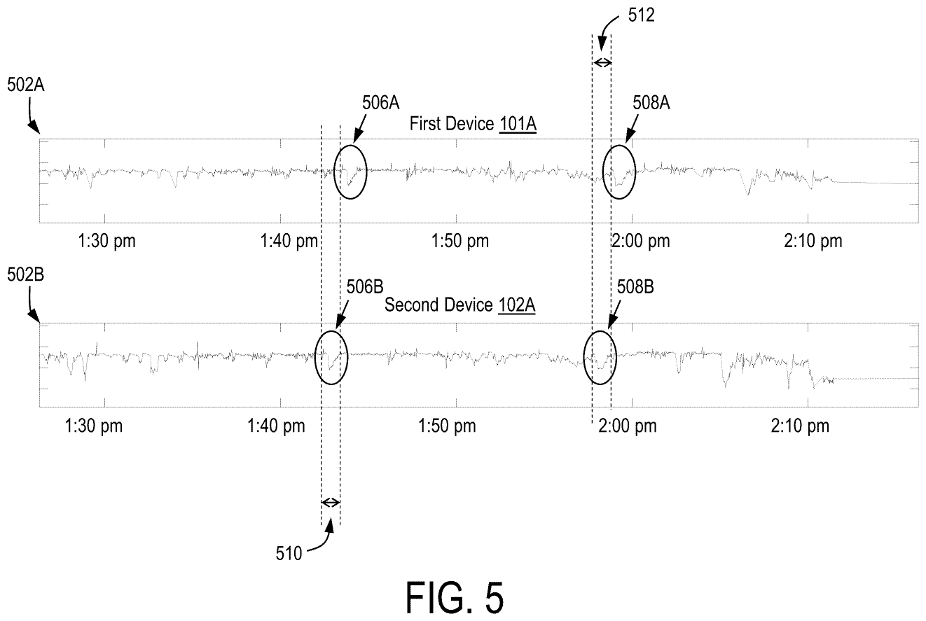

In embodiments, the abnormality location detection module 410 is configured to identify similar signal abnormalities in the time series (e.g., time series 415A, 415B, etc.) from different devices 101A, 102A. The signal abnormalities can be, for example, waveform patterns that can be compared across devices. For example, with reference to FIG. 5, two inter-beat-interval time series 502A, 502B are illustrated for an individual using two different devices 101A, 102A. As described herein, these time series 502A, 502B may be input to the abnormality location detection module 410, which may identify one or more abnormalities occurring in each time series 502A, 502B. As shown, a first abnormality 506A, 506B is identified in each time series 502A, 502B, but because the devices 101A, 102A are not synchronized to a common clock, there is a time-shift 510 that needs to be determined. In embodiments, more than one abnormality (e.g., at least a second abnormality 508A, 508B, etc.) may be identified as used to calculate one or more additional time shifts (e.g., time shift 512, etc.). Accordingly, in embodiments, the abnormality location detection module 410 is also configured to identify the relative timesteps of each of the signal abnormalities from the time series. The framework 400 can then include generating time-shifted signals 420 based on the input time series (e.g., time series 415A, 415B) based on the relative timesteps of the signal abnormalities. By determining a time-shift for the input time series 415A, 415B, other signals 430 (including different types of signals) measured by those devices 101A, 102A can then be synchronized based on the determined time-shift.

Once the measurements of the clinical study dataset 105 are synchronized, the method 100 can then include determining (in a step 130) data coverage metrics for the clinical study dataset 105 and/or determining (in a step 140) collection duration metrics for the clinical study dataset 105. It should be appreciated that step 130 may be performed before, after, or concurrently with step 140.

As described herein, a data coverage metric refers to whether measurements are available for a particular type of device 101A-D, 102A-D, 103A-D (or measurement channel of the device 101A-D, 102A-D, 103A-D, if a device 101A-D, 102A-D, 103A-D collects multiple types of measurements), that is being used for the clinical study. In embodiments, the data coverage metric can be a cross-sectional metric representative of data coverage across all subjects 100A-D of the clinical study. In such embodiments, for example, the data coverage metric may be a percentage that represents the number of subjects 100A-D for whom the clinical study dataset 105 includes measurements from a particular device 101A-D, 102A-D, 103A-D or device channel.

In embodiments, the data coverage metric can be a subject-specific metric representative of data coverage for a particular device 101A-D, 102A-D, 103A-D and a particular subject 100A-D. For example, in some embodiments, the data coverage metric may be a true/false determination depending on whether the clinical study dataset 105 includes measurements for a particular subject 100A-D from a particular device 101A-D, 102A-D, 103A-D or device channel.

In embodiments, the step 130 of the method 100 can include determining multiple data coverage metrics for the clinical study dataset 105 based on the synchronized measurements. For example, the step 130 can include determining one or more cross-sectional data coverage metrics for the clinical study dataset 105 and/or one or more patient-specific data coverage metrics.

In specific embodiments, the step 130 includes determining two or more data coverage metrics for the clinical study dataset 105 based on the synchronized measurements, wherein each data coverage metric corresponds to a particular device 101A-D, 102A-D, 103A-D. For example, in certain embodiments, the step 130 includes: (i) determining a first data coverage metric for a first device of two or more different devices 101A-D, 102A-D, 103A-D used in the clinical study, wherein the first data coverage metric corresponds to a percentage of the plurality of subjects 100A-D for whom the physiological information (i.e., the clinical study data 105) contains measurements collected using the first device; and (ii) determining a second data coverage metric for a second device of two or more different devices 101A-D, 102A-D, 103A-D, wherein the second data coverage metric corresponds to a percentage of the plurality of subjects 100A-D for whom the physiological information (i.e., the clinical study data 105) contains measurements collected using the second device.

In the step 140, the method 100 can also include determining one or more collection duration metrics for the clinical study dataset 105. As described herein, a collection duration metric refers to an evaluation of the period of time where measurements from a particular device 101A-D, 102A-D, 103A-D and/or device channel was collected for a particular subject 100A-D. In embodiments, the collection duration metric may be associated with a particular start time that corresponds to when the measurement was first collected and an end time that corresponds to when the measurement stopped being collected. For example, a plurality of collection duration metrics may be determined for a clinical study dataset 105, wherein the plurality of collection duration metrics includes a collection duration metric for each device 101A-D, 102A-D, 103A-D associated with each subject 100A-D.

In specific embodiments, the step 140 includes: (i) determining a first collection duration metric for a first device of two or more different devices 101A-D, 102A-D, 103A-D, wherein the first collection duration includes a first start time that corresponds to when the first device began collecting measurements from a subject 100A-D, and a first end time that corresponds to when the first device stopped collecting measurements from the subject 100A-D; and (ii) determining a second collection duration metric for a second device of the two or more different devices 101A-D, 102A-D, 103A-D, wherein the second collection duration includes a second start time that corresponds to when the second device began collecting measurements from a subject 100A-D, and a second end time that corresponds to when the second device stopped collecting measurements from the subject 100A-D.

In further embodiments, the step 140 includes: (i) determining a first collection duration metric for a first measurement or measurement type of the measurements included in the clinical study dataset 105, wherein the first collection duration metric includes a first start time that corresponds to when one of the devices 101A-D, 102A-D, 103A-D began collecting the first measurement from the subject 100A-D, and a first end time that corresponds to when the device 101A-D, 102A-D, 103A-D stopped collecting the first measurement; and (ii) determining a second collection duration metric for a second measurement or measurement type of the measurements included in the clinical study dataset 105, wherein the second collection duration metric includes a second start time that corresponds to when one of the devices 101A-D, 102A-D, 103A-D began collecting the second measurement from the subject 100A-D, and a second end time that corresponds to when the device 101A-D, 102A-D, 103A-D stopped collecting the second measurement.

In embodiments, one or more of the data coverage metrics and the collection duration metrics may be determined (in steps 130, 140) relative to the occurrence of at least one event of interest. In various embodiments, the event of interest can include, but is not limited to, the administration of a pharmaceutical compound to a subject 100A-D, the performance of a medical procedure or treatment in connection with the subject 100A-D, and/or the like.

In some embodiments, a data coverage metric may be determined as described above relative to the occurrence of at least one event of interest such that it is known whether data coverage exists within a predetermined time period relative to the at least one event of interest. For example, in a given clinical study, the event of interest may be the administration of an opioid prior to dental surgery, and seven different devices (e.g., devices 101A-D, 102A-D, 103A-D, etc.) are used to monitor each subject 100A-D before and/or after the event of interest. Thus, in such embodiments, the data coverage metrics may be determined such that it is known whether measurements from each of the seven different devices (e.g., devices 101A-D, 102A-D, 103A-D, etc.) are available before and/or after the event of interest.

In accordance with aspects of the present disclosure, the relevant time period for determining whether data coverage exists may be predetermined or otherwise customized based on the clinical study and/or adjusted by the user. For example, one clinical study may require six hours of measurements before and after the event of interest whereas another clinical study may require only measurements for 48 hours after the event of interest. Thus, in the former, the data coverage metric(s) would be determined by looking at the measurements available six hours before and after the event of interest, whereas in the latter, the data coverage metric(s) would be determined by looking at the measurements available within 48 hours after the event of interest.

In embodiments, the event of interest and the relevant time period may be selected by a user. Thus, in certain embodiments, the method 100 can include receiving user input identifying at least one event of interest and/or a relevant time period corresponding to an event of interest. In embodiments, the selection/user input may be received via an input device (discussed in more detail below). In embodiments, the selection/user input may be received prior to determining one or more data coverage metrics and/or collection duration metrics, after determining one or more data coverage metrics and/or collection duration metrics, and/or some combination of the before and after determining one or more data coverage metrics and/or collection duration metrics.

Next, in the step 150, the method 100 can include generating a dynamic user interface including the data coverage metrics and the collection duration metrics determined for the clinical study dataset 105. In particular embodiments, the dynamic user interface may be a dynamic graphical user interface comprising graphical representations of the data coverage metrics and/or the collection duration metrics.

In embodiments, the dynamic user interface may include a plurality of graphical modules configured to provide a graphical representation of one or more data coverage metrics and/or collection duration metrics for the clinical study dataset 105. For example, in some embodiments, the dynamic graphical user interface can include at least a first graphical module including a graphical representation of one or more data coverage metrics for a clinical study dataset 105, and at least a second graphical module including a graphical representation of one or more collection duration metrics for the clinical study dataset 105.

In further embodiments, the dynamic user interface may include at least a third graphical module including a graphical representation of the physiological information available for a plurality of subjects 100A-D stratified along a first axis corresponding to a start timestamp of the physiological information, and along a second axis corresponding to an end timestamp of the physiological information. In embodiments, the start and end timestamps may be offset relative to an event of interest, as described above. In further embodiments, one or more subject 100A-D may be represented via the third graphical module as a selectable indicator. That is, for example, selecting one of the indicators representative of a subject 100A-D updates the graphical interface to provide additional information about the selected subject 100A-D.

Next, in the step 160, the method 100 can include displaying the dynamic user interface on a display device of a system adapted to dynamically monitor incoming clinical study data.

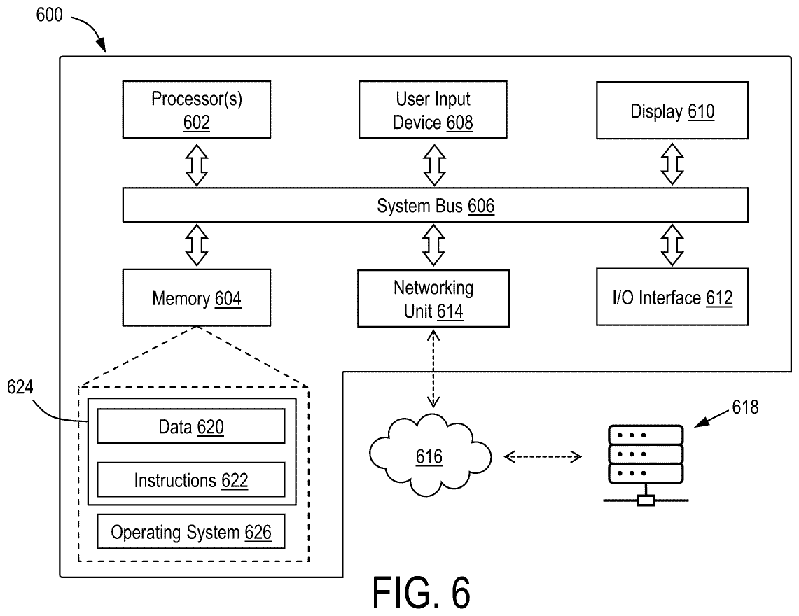

For example, with reference to FIG. 6, a system 600 for dynamically monitoring incoming clinical study data is illustrated in accordance with various aspects of the present disclosure. In embodiments, the system 600 includes: a display device 610 configured to display a dynamic graphical user interface; an input device 608 configured to receive user input; a computer-readable storage medium 604 having stored thereon computer-readable instructions 622 to be executed by one or more processors 602; and one or more processors 602 configured by the computer-readable instructions 622 stored on the computer-readable storage medium 604 to perform one or more operations of the methods described herein.

More specifically, in the example of FIG. 6, the system 600 can include one or more processors 602 and a computer-readable memory 604 interconnected and/or in communication via a system bus 606 containing conductive circuit pathways through which instructions (e.g., machine-readable signals) may travel to effectuate communication, tasks, storage, and the like. The system 600 can be connected to a power source (not shown), which can include an internal power supply and/or an external power supply. In embodiments, the system 600 can also include one or more additional components, such as a user input device 608, a display 610, an input/output (I/O) interface 612, a networking unit 614, and the like, including combinations thereof. As shown, each of these components may be interconnected and/or in communication via the system bus 606, for example.

In embodiments, the one or more processors 602 can include one or more high-speed data processors adequate to execute the program components described herein and/or perform one or more operations of the methods described herein. The one or more processors 602 may include a microprocessor, a multi-core processor, a multithreaded processor, an ultra-low voltage processor, an embedded processor, and/or the like, including combinations thereof. The one or more processors 602 can include multiple processor cores on a single die and/or may be a part of a system on a chip (SoC) in which the processor 602 and other components are formed into a single integrated circuit, or a single package. That is, the one or more processors 602 may be a single processor, multiple independent processors, or multiple processor cores on a single die.

In embodiments, the user input device 608 may be configured to receive various forms of input from a user associated with the system 600. The user input device 608 can include, but is not limited to, one or more of a keyboard, keypad, trackpad, trackball(s), capacitive keyboard, controller (e.g., a gaming controller), computer mouse, computer stylus/pen, a voice input device, and/or the like, including combinations thereof.

In embodiments, the display device 610 may be configured to display information, including text, graphs, and/or the like. In particular embodiments, the display device 610 may be configured to display a dynamic graphical user interface comprising one or more graphical modules that include graphical representations of data coverage metrics, collection duration metrics, and/or a combination thereof. The display device 610 can include, but is not limited to, a liquid crystal display (LCD), a light-emitting diode (LED) display, a touch screen or other touch-enabled display, a foldable display, a projection display, and so on, or combinations thereof.

In embodiments, the input/output (I/O) interface 612 may be configured to connect and/or enable communication with one or more peripheral devices (not shown), including but not limited to additional machine-readable memory devices, diagnostic equipment, and other attachable devices. The I/O interface 612 may include one or more I/O ports that provide a physical connection to the one or more peripheral devices. In some embodiments, the I/O interface 612 may include one or more serial ports.

In embodiments, the networking unit 614 may include one or more types of networking interfaces that facilitate wired and/or wireless communication between the system 600 and one or more external devices. That is, the networking unit 614 may operatively connect the system 600 to one or more types of communications networks 616, which can include a direction interconnection, the Internet, a local area network (“LAN”), a metropolitan area network (“MAN”), a wide area network (“WAN”), a wired or Ethernet connection, a wireless connection, a cellular network, and similar types of communications networks, including combinations thereof. In some embodiments, the system 600 may communicate with one or more remote/cloud-based servers and/or cloud-based services, such as remote server 618, via the communications network 616. In embodiments, the remote/cloud-based servers and/or cloud-based services can include, but are not limited to, one or more servers storing clinical study datasets 105 and/or intermediary platforms (e.g., data warehouse connection 104A, data loading platforms 104B, and/or the like).

In embodiments, the memory 604 can be variously embodied in one or more forms of machine accessible and machine-readable memory. In some embodiments, the memory 604 includes a storage device (not shown), which can include, but is not limited to, a non-transitory storage medium, a magnetic disk storage, an optical disk storage, an array of storage devices, a solid-state memory device, and/or the like, as well as combinations thereof. The memory 604 may also include one or more other types of memory, such as dynamic random-access memory (DRAM), static random-access memory (SRAM), erasable programmable read-only memory (EPROM), electrically erasable programmable read-only memory (EEPROM), Flash memory, and/or the like, as well as combinations thereof. In embodiments, the memory 604 may include one or more types of transitory and/or non-transitory memory.

The system 600 can be configured by software components stored in the memory 604 to perform one or more processes of the methods described herein. More specifically, the memory 604 can be configured to store data/information 620 and computer-readable instructions 622 that, when executed by the one or more processors 602, causes the system 600 to perform one or more operations of the methods described herein. Such data 620 and the computer-readable instructions 622 stored in the memory 604 may form a clinical study surveillance package 624 that may be incorporated into, loaded from, loaded onto, or otherwise operatively available to and from the system 600. Thus, in some embodiments, the clinical study surveillance package 624 and/or one or more individual software packages may be stored in a local storage device of the memory 604. However, in other embodiments, the clinical study surveillance package 624 and/or one or more individual software packages may be loaded onto and/or updated from a remote server or service, such as server 618, via the communications network 616. In particular embodiments, the clinical study surveillance package 624 can comprise a framework 400 for synchronizing signals from different devices, including an abnormality location detection module 410, as described above.

The system 600 may also include an operating system component 626, which may be stored in the memory 604. The operating system component 624 may be an executable program facilitating the operation of the system 600. Typically, the operating system component 626 can facilitate access of the I/O interface 612, network interface 614, the user input device 608, and the display 610, and can communicate or control other components of the system 600.

Accordingly, provided herein is a computer program product 624 comprising a non-transitory computer-readable storage medium 604 having stored thereon computer-readable instructions 622 that, when executed by one or more processors (such as processors 602), cause the one or more processors to perform one or more operations of the methods described below. For example, in specific embodiments, the computer-readable storage medium 604 may include computer-readable instructions 622 that, when executed by one or more processors (such as processors 602), cause the one or more processors to perform the following operations: (i) obtain a clinical study dataset comprising physiological information for a plurality of subjects over a period of time, wherein the physiological information includes two or more measurements collected from the plurality of subjects during the period of time using two or more different devices; (ii) analyze the clinical study dataset to synchronize the two or more measurements collected for each subject according to a common clock; (iii) determine two or more data coverage metrics for the clinical study dataset based on the synchronized measurements, wherein each data coverage metric corresponds to one of the two or more different devices; (iv) determine two or more collection duration metrics for the clinical study dataset based on the synchronized measurements, wherein each collection duration metric corresponds to either (a) one of the two or more different devices, or (b) one of the two or more measurements; (v) generate a dynamic graphical user interface including the two or more data coverage metrics and the two or more collection duration metrics determined for the clinical study dataset; and (vi) display, on a display device, the dynamic graphical user interface.

As such, the computer-readable storage medium 604 can include computer-readable instructions 622 that, when executed by one or more processors (such as processors 602), cause the one or more processors to perform a method for dynamically monitoring incoming clinical study data in accordance with the various aspects described herein.

For example, in particular embodiments, the computer-readable storage medium 604 can include computer-readable instructions 622 that, when executed by one or more processors (such as processors 602), cause the one or more processors to determine two or more data coverage metrics by: (i) determining a first data coverage metric for at least a first device of different devices 101A-D, 102A-D, 103A-D, wherein the first data coverage metric corresponds to a percentage of the plurality of subjects 100A-D for whom the physiological information contains measurements collected using the first device; and (ii) determining a second data coverage metric for at least second device of the different devices 101A-D, 102A-D, 103A-D, wherein the second data coverage metric corresponds to a percentage of the plurality of subjects 100A-D for whom the physiological information contains measurements collected using the second device.

In further embodiments, the computer-readable storage medium 604 can include computer-readable instructions 622 that, when executed by one or more processors (such as processors 602), cause the one or more processors to determine one or multiple duration coverage metrics by, for each subject of the plurality of subjects (e.g., subjects 100A-D): (i) determining a first collection duration metric for at least first device of the different devices 101A-D, 102A-D, 103A-D, wherein the first collection duration includes a first start time that corresponds to when the first device began collecting measurements from a corresponding subject, and a first end time that corresponds to when the first device stopped collecting measurements from that subject; and (ii) determining a second collection duration metric for at least a second device of the different devices 101A-D, 102A-D, 103A-D, wherein the second collection duration includes a second start time that corresponds to when the second device began collecting measurements from a corresponding subject, and a second end time that corresponds to when the second device stopped collecting measurements from that subject.

In embodiments, the computer-readable storage medium 604 can also include computer-readable instructions 622 that, when executed by one or more processors (such as processors 602), cause the one or more processors to: receive, via the input device 608, user input identifying at least one event of interest; and determine one or more data coverage metrics and/or collection duration metrics relative to the occurrence of the at least one event of interest identified by the user input.

As described herein, the dynamic graphical user interface can include one or more graphical modules including graphical representations of at least one data coverage metric and/or at least one collection duration metric. In embodiments, the dynamic graphical user interface may include at least a first graphical module including a graphical representation of one or multiple data coverage metrics for a clinical study dataset 105. In further embodiments, the dynamic graphical user interface may include at least a second graphical module including a graphical representation of the one or multiple collection duration metrics for a clinical study dataset 105.

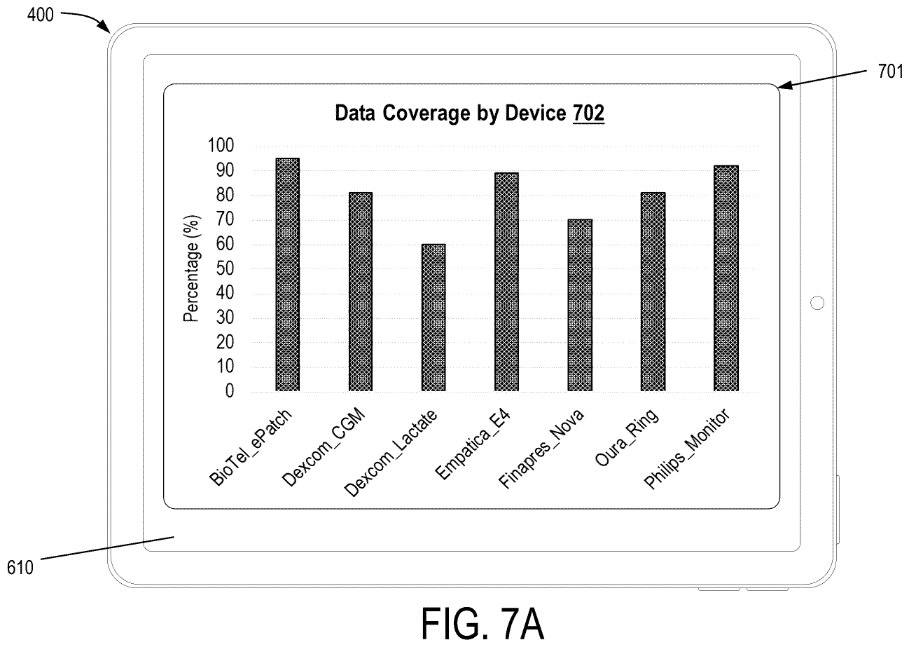

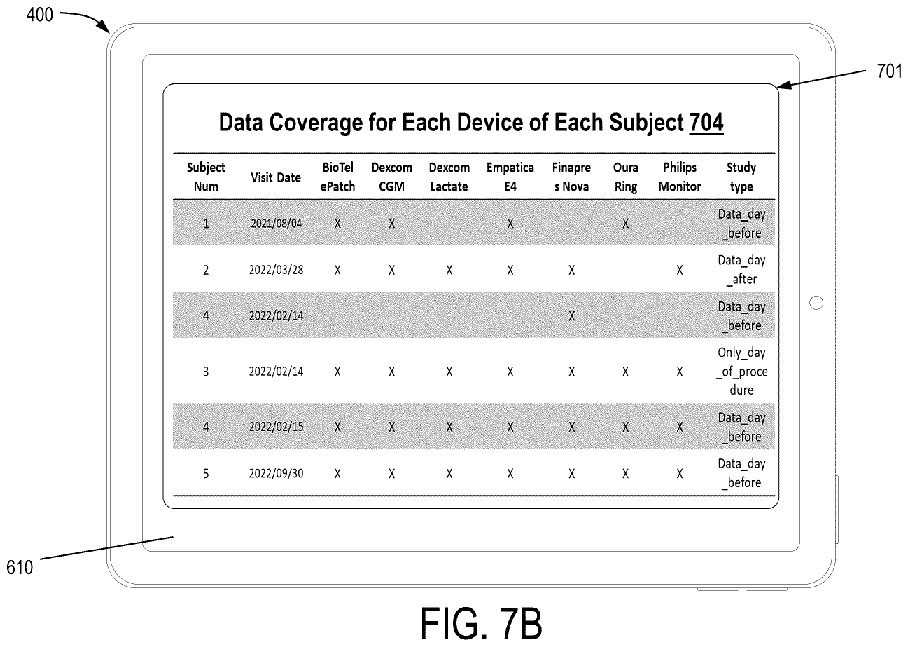

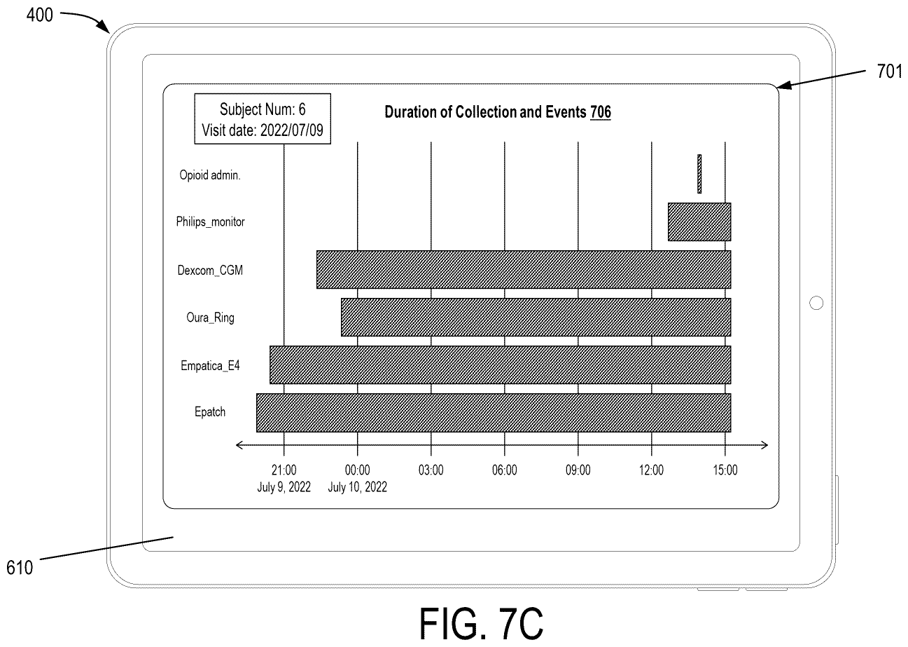

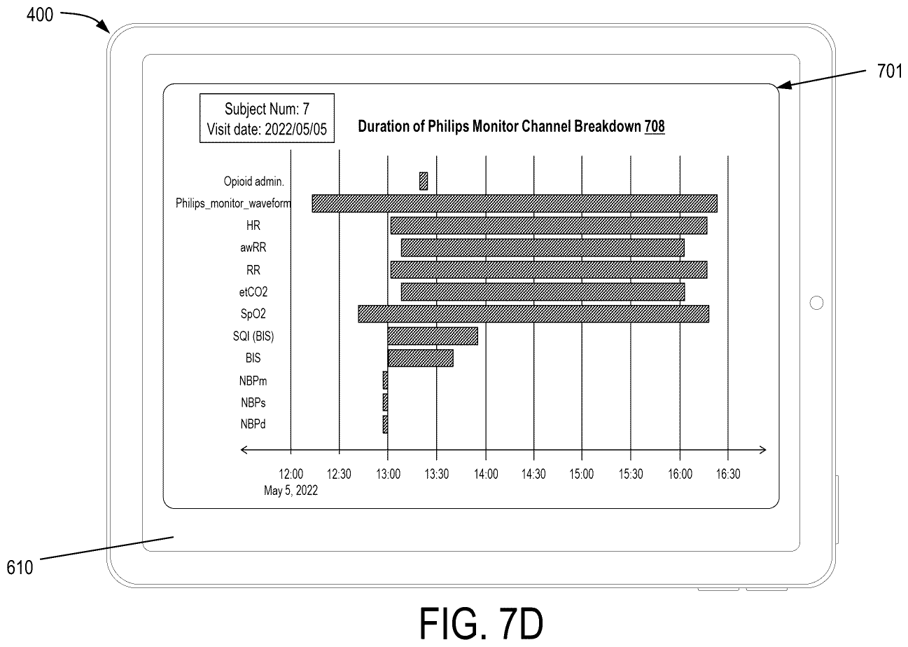

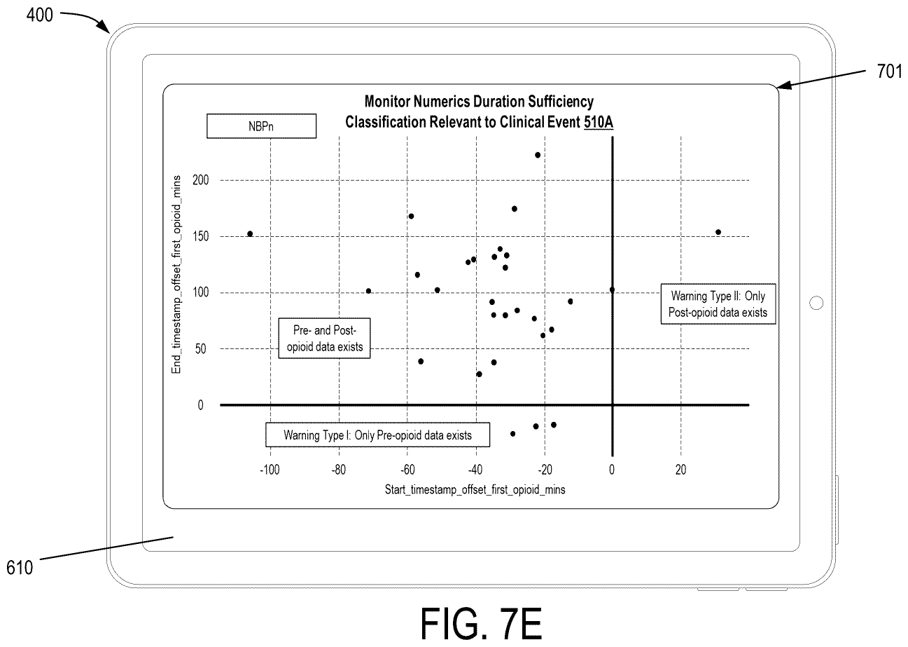

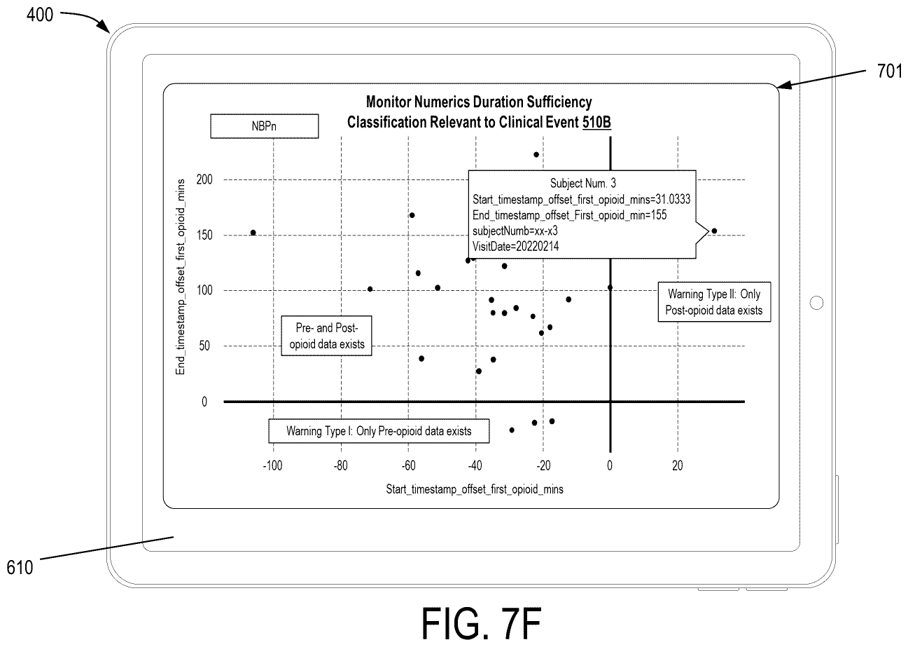

For example, with reference to FIGS. 7A-7D, a dynamic graphical user interface 701 having different graphical modules 702, 704, 706, 708 is illustrated in accordance with various aspects of the present disclosure. In the example of FIG. 7A, a system 600 comprising a display device 610 is illustrated with a first graphical module 702 of a dynamic graphical user interface 701 displayed thereon. As shown, the graphical module 702 includes multiple data coverage metrics for a clinical study dataset 105. In particular, the first graphical module 702 provides seven data coverage metrics for the clinical study dataset 105, which shows a percent data coverage for seven different devices (e.g., devices 101A-D, 102A-D, 103A-D, etc.) across all subjects (e.g., subjects 100A-D) of the clinical study dataset 105. Thus, as shown in the graphical module 702, the clinical study dataset 105 has over about 90% data coverage for the relevant time period across all subjects for the Bio-Tel ePatch measurements, but only has about 60% data coverage for the relevant time period(s) across all subjects for the Dexcom measurements.

In the example of FIG. 7B, the system 600 comprising a display device 610 is illustrated with another graphical module 704 of a dynamic graphical user interface 701 displayed thereon. As shown, the graphical module 704 includes multiple data cover metrics for a plurality of patients within a clinical study dataset 105. The graphical module 704 provides a plurality of true/false data coverage indicators (represented by “x” marks, which indicate the existence of data for the given patient and the given device). Thus, for example, Subject 1 has data coverage for four different devices over a particular time period, but is missing data coverage for three other devices over the same time period. In this graphical module 704, the “study type” and the “visit date” can also be displayed. In another example, Subject 4 (on one particular visit) only has data coverage for one device and is missing data coverage for the other six different devices.

In the example of FIG. 7C, the system 600 comprising a display device 610 is illustrated with yet another graphical module 706 of a dynamic graphical user interface 701 displayed thereon. As shown, the graphical module 706 includes multiple collection duration metrics for multiple devices (e.g., devices 101A-D, 102A-D, 103A-D) and for a specific patient within a clinical study data 105. The graphical module 706 provides the collection duration metrics as horizontal bars extending between a start time and an end time for each device 101A-D, 102A-D, 103A-D. Thus, for example, the graphical module 706 illustrates the start and end times of data collection for five different devices (e.g., devices 101A-D, 102A-D, 103A-D). The graphical module 706 also illustrates the occurrence of an event of interest, namely, the administration of an opioid to the subject. Notably, the collection duration metrics shown in the graphical module 706 also make is easy to ascertain whether certain data coverage is available (e.g., before and after the event of interest, etc.).

Similarly, in the example of FIG. 7D, the graphical module 708 includes multiple collection duration metrics for different channels of a single device (e.g., a Philips monitor, etc.). As described above, it is possible that a single device may collect more than one type of physiological information. As shown in FIG. 7D, the Philips monitor collects, for example, a plurality of different measurements, each of which may have their own collection duration metric. That is, as shown, the different channels of a particular device may have start and end times to the data collection, and therefore have different collection duration metrics.

It should be appreciated that the graphical modules 706, 708 shown in FIGS. 7D and 7E may be generated for each subject of the clinical study data 105. Thus, although the graphical modules 706, 708 are shown in connection with just one subject, it should be understood that graphical modules 704, 706 may be generated for the other subjects as well. Further, with respect to the graphical modules 702, 704, 706, 708, the data coverage metrics and the collection duration metrics may be determined in relation to a particular event of interest, as described herein.

In still further embodiments, the dynamic graphical user interface may include at least a third graphical module including a graphical representation of the physiological information available for one or multiple subjects stratified along different axes. For example, in embodiments, the physiological information may be stratified along a first axis corresponding to a start timestamp of the physiological information, and along a second axis corresponding to an end timestamp of the physiological information. In some embodiments, the start and/or end timestamps may be offset relative to an event of interest. In further embodiments, one or more subjects 100A-D of the clinical study data 105 can be represented in the third graphical module by a selectable indicator such that selecting a particular subject 100A-D causes the graphical module to present detailed information for the selected subject 100A-D. Put another way, the computer-readable storage medium 604 can include computer-readable instructions 622 that, when executed by one or more processors (such as processors 602), further cause the one or more processors to: receive a user selection of a selectable indicator corresponding to a selected subject; and, update the graphical module to present detailed information for the selected subject, wherein the detailed information includes physiological information not otherwise represented in the third graphical module.

For example, with reference to FIGS. 7E-7F, a dynamic graphical user interface 701 having a third type of graphical module 710A, 710B is illustrated in accordance with various aspects of the present disclosure. As shown in FIG. 7E, the graphical module 710A comprises a graphical representation of the availability of physiological information (i.e., NBPn measurements) for multiple subjects stratified along a first axis corresponding to a start timestamp of the physiological information, and along a second axis corresponding to an end timestamp of the physiological information. In embodiments, the start and/or end timestamps may be offset relative to an event of interest. For example, in FIG. 7E, the start and end timestamps are offset based on the known timing of the administration of an opioid to the subject. Thus, subjects in the first quadrant of the graphical module 710A as be identified as having data coverage for the given device and/or device channel both before and after the opioid was administered. In the same manner, subjects in the second quadrant can be readily identified as having only post-opioid data coverage, while subjects in the third quadrant can be readily identified as having only pre-opioid data for the given device and/or device channel.

As described above, the one or more subjects 100A-D of the clinical study data 105 can be represented in the third graphical module 710A by a selectable indicator such that selecting a particular subject 100A-D causes the graphical module to present detailed information for the selected subject 100A-D. As shown in FIG. 7E, each subject is represented by a selectable dot. When a user input selecting one of the subjects is received, the graphical module 710A may be updated to present detailed information for the selected subject. For example, as shown in FIG. 7F, Subject 3 is selected by the user and the graphical module 710B is updated to display additional information for the subject.

In accordance with various aspects of the present disclosure, the systems, platforms, and methods described herein provide actionable, real-time insight into data collection integrity of a clinical study involving a plurality of subjects and multiple different modalities/devices. These systems, platforms, and methods further enable selection and customization based on the occurrence of a specific event or type of event, such as the administration of a pharmaceutical compound before/after a medical procedure. As such, the systems, platforms, and methods provided herein address unique technical challenges important to the credibility and robustness of a clinical study. The systems, platforms, and methods can further help identify previously unknown sources of error that lead to gaps in data coverage and/or gaps in collection duration. Additionally, the systems, platforms, and methods described herein enable and enhance communication between those responsible for analyzing the clinical study data (e.g., the data scientists, etc.) and those responsible for collecting the data (e.g., the physicians and clinical staff, etc.).

Thus, in accordance with various embodiments, the present disclosure enables data scientists to interpret a model's confidence and variance depending on whether the clinical study shows small missing data versus large missing data (where a small amount of missing data would indicate that model results have high confidence and lower variance, and vice versa). The present disclosure also enables feature selection whereby a user may select a set of features with a low missing rate based on the system. For example, if glucose data has a high missing rate for most subjects, a data scientist can remove them. The present disclosure further enables feature selection based on collection duration metrics because duration is calculated relative to the event of interest (intervention, for example), which makes it easy for data scientists to select features based on availability relevant to the event. For example, if the modeling task requires some amount of pre-event data and some amount of post-event data, data scientists can make the choice of feature based on availability specific to those requirements. In still further aspects, the present disclosure enables cohort selection whereby data scientists can make a decision on cohort selection based on how complete the data coverage is versus how many gaps there are based on the dynamic user interface.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method of dynamically monitoring incoming clinical study data, the method comprising:
obtaining a clinical study dataset comprising physiological information for a plurality of subjects over a period of time, wherein the physiological information includes two or more measurements collected from the plurality of subjects during the period of time using two or more different devices;
analyzing the clinical study dataset to synchronize the two or more measurements collected for each subject according to a common clock;
determining two or more data coverage metrics for the clinical study dataset based on the synchronized measurements, wherein each data coverage metric corresponds to one of the two or more different devices;
determining two or more collection duration metrics for the clinical study dataset based on the synchronized measurements, wherein each collection duration metric corresponds to either (i) one of the two or more different devices, or (ii) one of the two or more measurements;
generating a dynamic graphical user interface including the two or more data coverage metrics and the two or more collection duration metrics determined for the clinical study dataset;
displaying, on a display device, the dynamic graphical user interface.

2. The method of claim 1, wherein determining two or more data coverage metrics includes:
determining a first data coverage metric for a first device of the two or more different devices, wherein the first data coverage metric corresponds to a percentage of the plurality of subjects for whom the physiological information contains measurements collected using the first device; and
determining a second data coverage metric for a second device of the two or more different devices, wherein the second data coverage metric corresponds to a percentage of the plurality of subjects for whom the physiological information contains measurements collected using the second device.

3. The method of claim 1, wherein determining two or more duration coverage metrics includes, for each subject of the plurality of subjects:
determining a first collection duration metric for a first device of the two or more different devices, wherein the first collection duration metric includes a first start time that corresponds to when the first device began collecting measurements from the subject, and a first end time that corresponds to when the first device stopped collecting measurements from the subject; and
determining a second collection duration metric for a second device of the two or more different devices, wherein the second collection duration includes a second start time that corresponds to when the second device began collecting measurements from the subject, and a second end time that corresponds to when the second device stopped collecting measurements from the subject.

4. The method of claim 1, wherein determining two or more duration coverage metrics includes, for each subject of the plurality of subjects:
determining a first collection duration metric for a first measurement of the two or more measurements, wherein the first collection duration metric includes a first start time that corresponds to when one of the two or more different devices began collecting the first measurement from the subject, and a first end time that corresponds to when the device stopped collecting the first measurement; and
determining a second collection duration metric for a second measurement of the two or more measurements, wherein the second collection duration metric includes a second start time that corresponds to when one of the two or more different devices began collecting the second measurement from the subject, and a second end time that corresponds to when the device stopped collecting the second measurement.

5. The method of claim 4, wherein the same device is used to collect the first measurement and the second measurement.

6. The method of claim 1, wherein the two or more data coverage metrics and the two or more collection duration metrics are determined relative to the occurrence of at least one event of interest.

7. The method of claim 6, further comprising:
receiving, via an input device, user input identifying the at least one event of interest.

8. A system for dynamically monitoring incoming clinical study data, the system comprising:
a display device configured to display a dynamic graphical user interface;
an input device configured to receive user input;
a computer-readable storage medium having stored thereon computer-readable instructions to be executed by one or more processors; and
one or more processors configured by the computer-readable instructions stored on the computer-readable storage medium to perform the following operations: (i) obtain a clinical study dataset comprising physiological information for a plurality of subjects over a period of time, wherein the physiological information includes two or more measurements collected from the plurality of subjects during the period of time using two or more different devices; (ii) analyze the clinical study dataset to synchronize the two or more measurements collected for each subject according to a common clock; (iii) determine two or more data coverage metrics for the clinical study dataset based on the synchronized measurements, wherein each data coverage metric corresponds to one of the two or more different devices; (iv) determine two or more collection duration metrics for the clinical study dataset based on the synchronized measurements, wherein each collection duration metric corresponds to either (a) one of the two or more different devices, or (b) one of the two or more measurements; (v) generate a dynamic graphical user interface including the two or more data coverage metrics and the two or more collection duration metrics determined for the clinical study dataset; and (vi) display, on a display device, the dynamic graphical user interface.

9. The system of claim 8, wherein the one or more processors are further configured by the computer-readable instructions stored on the computer-readable storage medium to perform the following operations:
receive, via the input device, user input identifying at least one event of interest; and
determine the two or more data coverage metrics and the two or more collection duration metrics relative to the occurrence of the at least one event of interest.

10. The system of claim 8, wherein the dynamic graphical user interface comprises:
a first graphical module including a graphical representation of the two or more data coverage metrics for the clinical study dataset; and
a second graphical module including a graphical representation of the two or more collection duration metrics for the clinical study dataset.

11. The system of claim 10, wherein the dynamic graphical user interface comprises:
a third graphical module including a graphical representation of the physiological information available for the plurality of subjects stratified along a first axis corresponding to a start timestamp of the physiological information offset relative to an event of interest, and along a second axis corresponding to an end timestamp of the physiological information offset relative to the event of interest, wherein each subject is represented by a selectable indicator.

12. The system of claim 11, wherein the one or more processors are further configured by the computer-readable instructions stored on the computer-readable storage medium to perform the following operations:
receive, via the input device, a user selection of a selectable indicator corresponding to a selected subject; and
updating the third graphical module to present detailed information for the selected subject, wherein the detailed information includes physiological information for the selected subject not represented in the third graphical module.

13. A computer program product comprising:
a computer-readable storage medium having stored thereon computer-readable instructions that, when executed by one or more processors, cause the one or more processors to perform the following operations: (i) obtain a clinical study dataset comprising physiological information for a plurality of subjects over a period of time, wherein the physiological information includes two or more measurements collected from the plurality of subjects during the period of time using two or more different devices; (ii) analyze the clinical study dataset to synchronize the two or more measurements collected for each subject according to a common clock; (iii) determine two or more data coverage metrics for the clinical study dataset based on the synchronized measurements, wherein each data coverage metric corresponds to one of the two or more different devices; (iv) determine two or more collection duration metrics for the clinical study dataset based on the synchronized measurements, wherein each collection duration metric corresponds to either (a) one of the two or more different devices, or (b) one of the two or more measurements; (v) generate a dynamic graphical user interface including the two or more data coverage metrics and the two or more collection duration metrics determined for the clinical study dataset; and (vi) display, on a display device, the dynamic graphical user interface.

14. The computer program product of claim 13, wherein the computer-readable storage medium includes computer-readable instructions to determine two or more data coverage metrics by:
determine a first data coverage metric for a first device of the two or more different devices, wherein the first data coverage metric corresponds to a percentage of the plurality of subjects for whom the physiological information contains measurements collected using the first device; and
determine a second data coverage metric for a second device of the two or more different devices, wherein the second data coverage metric corresponds to a percentage of the plurality of subjects for whom the physiological information contains measurements collected using the second device.

15. The computer program product of claim 13, wherein the computer-readable storage medium includes computer-readable instructions to determine two or more duration coverage metrics by, for each subject of the plurality of subjects:
determine a first collection duration metric for a first device of the two or more different devices, wherein the first collection duration includes a first start time that corresponds to when the first device began collecting measurements from the subject, and a first end time that corresponds to when the first device stopped collecting measurements from the subject; and
determine a second collection duration metric for a second device of the two or more different devices, wherein the second collection duration includes a second start time that corresponds to when the second device began collecting measurements from the subject, and a second end time that corresponds to when the second device stopped collecting measurements from the subject.
